(19) United States
(12) Patent Application Publication
(10) Pub. No.: US 2020/0375891 A1
(43) Pub. Date: Dec. 3, 2020

(54) BIOERODIBLE CROSS-LINKED HYDROGEL IMPLANTS AND RELATED METHODS OF USE

(71) Applicant: Dose Medical Corporation, San Clemente, CA (US)

(72) Inventors: Patrick Michael Hughes, San Clemente, CA (US); David Bardin, San Clemente, CA (US); Ina Mustafaj, San Clemente, CA (US); Harold A. Heitzmann, San Clemente, CA (US)

(21) Appl. No.: 16/888,348

(22) Filed: May 29, 2020

Related U.S. Application Data
(60) Provisional application No. 62/855,647, filed on May 31, 2019.

(51) Int. Cl. A61K 9/00; A61K 47/10; A61K 47/12; A61K 39/395; A61K 38/17

(52) U.S. Cl. CPC A61K 9/0024; A61K 9/0051; A61K 38/179; A61K 47/12; A61K 39/3955; A61K 47/10

(57) ABSTRACT

The present disclosure is directed to a composite implant for the sustained release of a therapeutic agent from a hydrogel matrix. The hydrogel matrix may be a cross-linked bioerodible polyethylene glycol (PEG) hydrogel with a therapeutic complex dispersed within the cross-linked bioerodible PEG hydrogel. The therapeutic complex may include a therapeutic agent in association with either a fatty acid or fatty alcohol and/or any other excipients, peptides, or nucleic acids. The composite implant is configured to be delivered to or implanted into an eye of a subject or patient. The composite implant may comprise a rod shape. The composite implant may be used treat ocular disease in a subject or patient. Ocular diseases may be selected from at least one of neovascular age related macular degeneration (AMD), diabetic macular edema, or macular edema following retinal vein occlusion.

### RELATED APPLICATIONS

This application claims priority to U.S. Provisional Patent Application No. 62/855,647, filed May 31, 2019, and titled BIOERODIBLE CROSS-LINKED HYDROGEL IMPLANTS AND RELATED METHODS OF USE, which is incorporated herein by reference in its entirety.

### TECHNICAL FIELD

The present disclosure relates to composite implants for treating ocular diseases, such as neovascular age-related macular degeneration (AMD), diabetic macular edema, and macular edema following retinal vein occlusion. In particular, the composite implants include a composition that provides sustained release of a therapeutic complex from a composite bioerodible hydrogel matrix. The present disclosure further relates to methods for making and manufacturing bioerodible cross-linked hydrogel implants, as well as related methods of using the bioerodible cross-linked hydrogel implants.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 illustrates a schematic of a cross-linked composite implant with a cross-linked polymer with a degradable linkage and a therapeutic complex, according to one embodiment.

FIG. 2 illustrates an 8-arm polyethylene glycol-succinimidyl glutarate (PEG-SG), which constitutes a component of a cross-linked hydrogel, according to one embodiment.

FIG. 3 illustrates an R group for a polyethylene glycol-succinimidyl glutarate (PEG-SG), which constitutes a component of a cross-linked hydrogel, according to one embodiment.

FIG. 4 illustrates an R group for polyethylene glycol-succinimidyl adipate (PEG-SAP), which constitutes a component of a cross-linked hydrogel, according to one embodiment.

FIG. 5 illustrates an 8-arm polyethylene glycol electrophilic end group (PEG-NH2), which constitutes a component of a cross-linked hydrogel, according to one embodiment.

FIG. 6A illustrates a cross-linking reaction via the illustrated mechanism, according to one embodiment.

FIG. 6B illustrates a cross-linking reaction via the illustrated mechanism, according to one embodiment.

FIG. 7 is a graph showing in vitro release of bevacizumab from PEG-SG into isotonic phosphate buffered saline, pH 7.4 at 37° C. and 45° C., according to one embodiment.

FIG. 8 is a graph showing in vitro release of bevacizumab from PEG-SG into isotonic phosphate buffered saline, pH 7.4 at 37° C. and 40° C., according to one embodiment.

FIG. 9 is a graph showing in vitro release of aflibercept from PEG-SG into isotonic phosphate buffered saline, pH 7.4 at 37° C. and 40° C., according to one embodiment.

FIG. 10 illustrates a plurality of composite aflibercept PEG hydrogel implants with fatty alcohol, according to one embodiment.

FIG. 11 illustrates aflibercept release from composite PEG-SG/fatty alcohol hydrogels, according to one embodiment.

FIG. 12 illustrates aflibercept release from composite PEG-SAP/fatty alcohol hydrogels, according to one embodiment.

### DETAILED DESCRIPTION

Proteins are attractive therapeutic targets due to their specificity and potency. Biologics such as proteins are becoming increasingly important in medicine. In ophthalmology, several biologics have had tremendous therapeutic impact. Bevacizumab, ranibizumab, and aflibercept are examples of proteins that have been shown to provide great clinical benefit in subjects having diseases such as neovascular age-related macular degeneration (AMD) and diabetic macular edema.

Proteins are hydrophilic, water soluble macromolecules that have poor membrane permeation. As such, the bioavailability of proteins from oral administration or topical administration is poor. To circumvent the absorption barriers for proteins, they are usually administered by parenteral administration or direct injection into the desired biologic compartment, such as intraocular administration. There are several constraints to productive absorption of therapeutic proteins into the eye. Topically, macromolecules such as proteins will have limited permeability to the corneal epithelium and a rapid pre-corneal clearance from topical dosing. Typically, only 1% to 5% of a topically administered small molecule eye drop is bioavailable to the aqueous humor. Topical bioavailability of proteins is considerably less. Further movement to the posterior segment of the eye is limited by the iridolenticular diaphragm and the diffusional barrier presented by the vitreous. Hence, little to no topically applied drug can reach the posterior segment of the eye by the macula. The blood-retinal barriers and blood-aqueous barriers further prevent intraocular uptake of proteins from systemic administration. The proteins to be administered require intravitreal injection to achieve therapeutic concentrations in the posterior segment of the eye.

Proteins also suffer from rapid clearance from the systemic circulation. While the clearance of proteins from the vitreous is slower, with half-lives on the order of days, they are still cleared rapidly relative to the duration of therapy. This requires proteins to be injected into the eye at high concentrations to prolong therapeutic effect and by frequent monthly or bi-monthly injections. The result is transient high initial intraocular concentrations of the protein, which can lead to unintended side effects and frequent intravitreal injections that increases the risk for endophthalmitis, cataract, retinal detachment, and other detrimental sequelae.

Therefore, sustained delivery of proteins directly to the intraocular space would greatly improve the therapeutic benefit to patients. Despite the high medical value of sustained protein delivery systems to the eye, no system has been successfully developed. Sustained delivery of proteins offers several unique challenges. Proteins are sensitive to aggregation and potential immunogenicity issues, denaturation, and loss of activity and degradation. This can be brought about by the sheer and thermal stresses encountered during manufacturing, aggregation and degradation in aqueous environments, loss of tertiary and quaternary structure and activity, and losses to interfaces. Most proteins are sensitive to extremes of pH. Poly-lactide-co-glycolide, polycaprolactone, and other polyester bioerodible polymers are commonly used to formulate sustained release delivery systems. Unfortunately, proteins can degrade, aggregate, or lose activity at the hydrophobic interfaces during manufacture of the delivery system, upon hydration of the delivery systems and protein release, or in the acidic microenvironment created as these polymers degrade in vivo.

Hydrogels provide an attractive alternative to polyesters because they create a protein friendly environment and acidic products of degradation can diffuse away prior to affecting the protein. However, hydrogels have a high water content that can cause protein degradation and aggregation. Hydrogels are also relatively porous, rendering it difficult to control the protein release. Our work has shown that cross-linked PEG hydrogels, by themselves, do not provide sustained protein release beyond a few months and that protein aggregates and degrades within 30 days in an aqueous environment. Further, the use of fatty alcohol particulates on their own also do not provide sustained protein release. Notably, stearic acid and stearyl alcohol protein particulates released all of the protein within one day. Surprisingly and unexpectedly, a composite system of (i) a cross-linked bioerodible PEG hydrogel and (ii) therapeutic complexes of aflibercept associated with fatty alcohol dispersed in the cross-linked bioerodible PEG hydrogel enables sustained release of aflibercept for several months, while maintaining the stability of the released protein.

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

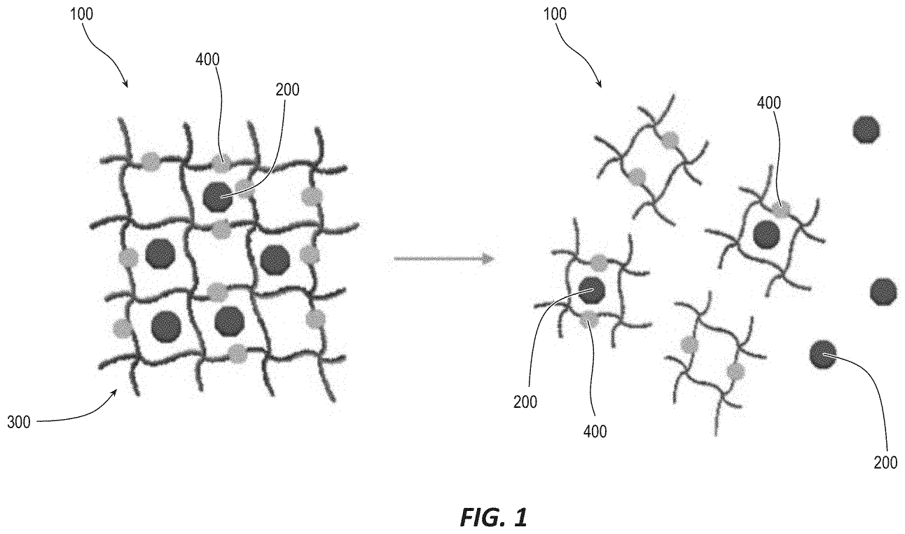

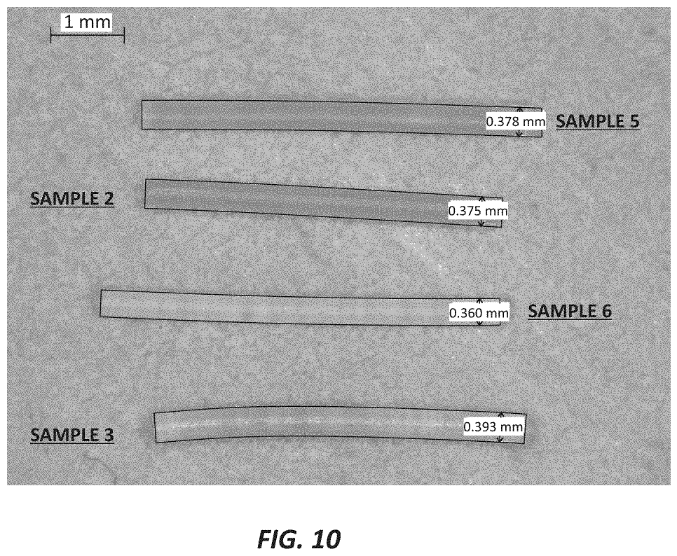

FIG. 1 provides a schematic illustration of a portion of a composite implant 100 for the sustained release of a therapeutic agent 200 from a hydrogel matrix 300. The hydrogel matrix 300 may include degradable linkages 400 that enable the release of the therapeutic agent 200 from the hydrogel matrix 300 over time. In some embodiments, the hydrogel matrix 300 may be a cross-linked bioerodible polyethylene glycol (PEG) hydrogel with a therapeutic complex dispersed within the cross-linked bioerodible PEG hydrogel. The therapeutic complex may include a therapeutic agent 200 in association with either a fatty acid or fatty alcohol and/or any other excipients, peptides, or nucleic acids. The composite implant 100 may be configured to be delivered to or implanted into an eye of a subject or a patient. The composite implant 100 may comprise a rod shape, as illustrated, for example, in FIG. 10.

The composite implant may be used to treat ocular diseases in a subject or a patient. Ocular diseases may be selected from at least one of neovascular age-related macular degeneration, diabetic macular edema, and macular edema following retinal vein occlusion. Other ocular diseases that may be treated by the composite implant include, but are not limited to, proliferative vitreal retinopathy, dry AMD, glaucoma (neuroprotection), uveitis, vitritis, endophthalmitis, infection, inflammation, cataract, retinitis pigmentosa, chorioretinitis, choroiditis, and autoimmune disorders.

The therapeutic complex may include a therapeutic agent associated with a fatty component, such as a fatty alcohol, fatty acid, or a fatty alcohol/fatty acid blend matrix. In particular embodiments, the association between the therapeutic agent and the fatty component in the therapeutic complex can be achieved by various means, such as hot melt extrusion, blending, compression, granulation, roller compaction, spray drying, co-lyophilization, spray freeze drying, microencapsulation, melt encapsulation, coacervation, solvent casting, microfluidics, injection molding, and/or other method for fabricating microparticles and the like. In certain embodiments, the therapeutic complex may be composed of the therapeutic agent dispersed within, coated by, and/or adsorbed to the fatty component. The therapeutic agent may be at least one of a protein, a peptide, a nucleic acid, an RNA, an siRNA, apatamers such as pegaptanib, or a small molecule. The therapeutic agent may include at least one of a prostaglandin, a neuroprotectant, a retinoid, squalamine, a steroid, an alpha adrenergic agent, a gene, an antibiotic, a non-steroidal anti-inflammatory agent, a calcineurin inhibitor such as cyclosporine, an adeno-associated virus vector, a tyrosine kinase inhibitor, or a rho kinase inhibitor.

The therapeutic protein/peptide may include, but is not limited to, bevacizumab, ranibizumab, aflibercept, brolucizumab, faricimab, conbercept (recombinant anti-VEGF fusion protein), ankyrin repeat proteins such as abicipar pegol, adalimumab and other anti-TNF-alpha agents, biosimilars, their respective salts, esters, solvates, isomers, or complexes, and conjugates such as pegylation.

The hydrogel serves to sequester the therapeutic complexes and to modulate the release of the protein from the implant. The therapeutic complexes, formed by the association of a therapeutic agent and a fatty component, serve to stabilize the therapeutic agent to manufacturing processes and the aqueous environment in vivo during release as well as to provide a sustained or controlled release of the therapeutic agent. In addition, pharmaceutically acceptable ingredients such as excipients, release modifiers, and surfactants among others may be incorporated into the composition.

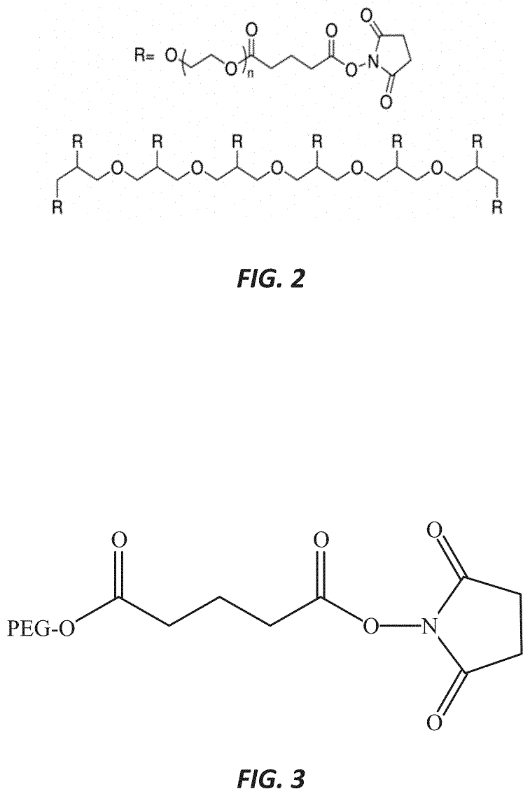

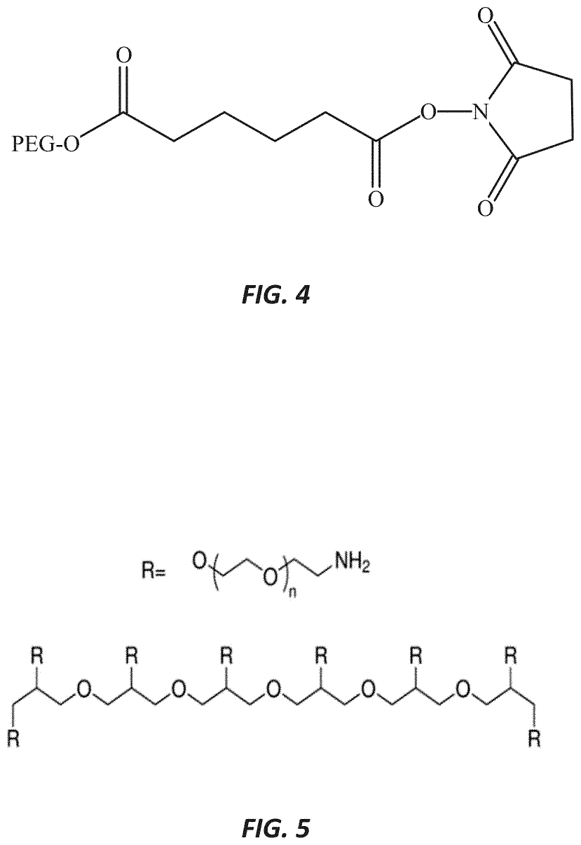

The PEG component of the composite implant comprises a PEG with an electrophilic end group (PEG-NHS) and a PEG with a nucleophilic end group (PEG-NH2). The electrophilic PEG can be selected from the group consisting of different chain lengths and different cores (e.g., hexaglycerol and pentaerythritol). In some embodiments the PEG-NHS group includes electrophilic groups such as SG (N-hydroxysuccinimidyl glutarate), SAP (N-hydroxysuccinimidyl adipate), and SAZ (N-hydroxysuccinimidyl azelate). FIG. 2 illustrates an 8-arm polyethylene glycol-succinimidyl glutarate (PEG-SG), FIG. 3 illustrates an R group for a polyethylene glycol-succinimidyl glutarate (PEG-SG), and FIG. 4 illustrates an R group for polyethylene glycol-succinimidyl adipate (PEG-SAP). A difference in the number of methylene units (spacers) between the functional group and the core can affect degradation and release: SG=3 spacers, SAP=4 spacers, and SAZ=7 spacers. The electrophilic and hydrophilic PEG groups cross-link to form a mesh-like delivery system as depicted in FIG. 1. FIG. 5 illustrates an exemplary embodiment of an 8-arm PEG-NH2.

The hydrogel may be optimized for mesh size (opening between cross-linking), therapeutic agent release rate, and erosion kinetics by varying the cross-linking density (4-arm, 6-arm, and 8-arm PEGs), linker chain length (longer chains=slower hydrolysis and slower release), and PEG molecular weight (the higher molecular weight, the larger the pore size and the faster the release). The PEG core may also be optimized to alter cross-linking and erosion.

The therapeutic complex is composed of a therapeutic agent (i.e., therapeutic protein, etc.) associated with a fatty component (i.e., fatty alcohol, fatty acid, fatty alcohol/fatty acid blend matrix, etc.). The therapeutic agent may be associated with the fatty component by, for example, but not to be limited to, being: 1) dispersed within the fatty alcohol, fatty acid, or fatty alcohol/fatty acid blend matrix; 2) coated by the fatty alcohol, fatty acid or fatty alcohol/fatty acid blend; 3) adsorbed to the fatty alcohol, fatty acid or fatty alcohol/fatty acid blend, or the fatty alcohol, fatty acid or fatty alcohol/fatty acid blend adsorbed to the therapeutic agent; or 4) any combination thereof. The association between the therapeutic agent and the fatty component may be achieved and the therapeutic complex may be fabricated by hot melt extrusion, blending, compression, granulation, roller compaction, spray drying, co-lyophilizing, spray freeze drying, microencapsulation, melt encapsulation, coacervation, solvent casting, microfluidics, injection molding, and any other technique for fabricating complexes or microparticles known in the art. Other materials suitable for the complex material may include polyanhydrides and poly(ortho esters).

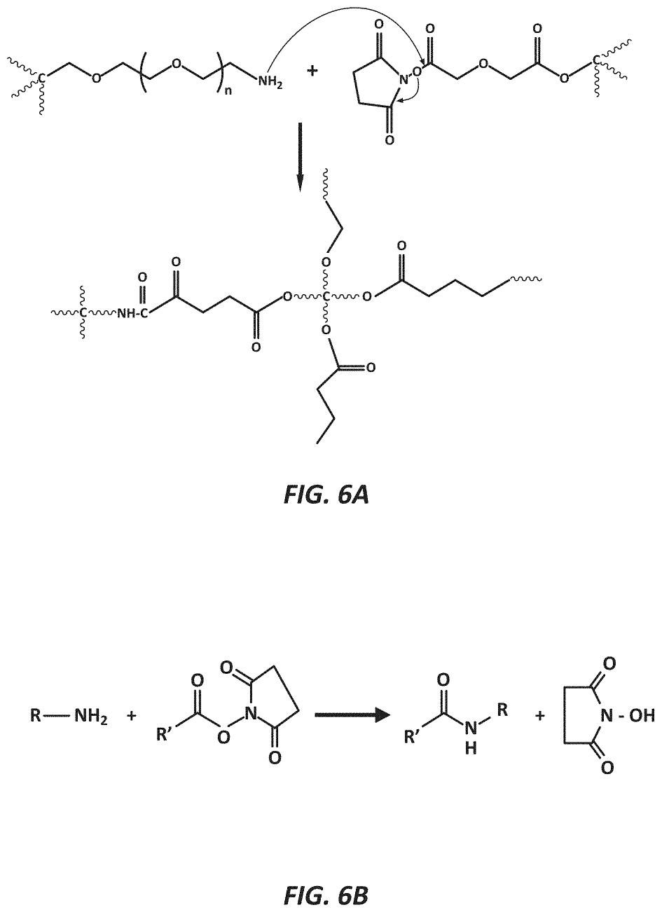

The hydrogel is manufactured by dissolving the PEG-NHS in dichloromethane (DCM) or water in a vial. The PEG-NH2 is then dissolved in DCM or water in a separate vial and a formulation comprising the therapeutic agent (e.g., a therapeutic protein) is added. The two vials may be mixed with or without triethylamine to catalyze a reaction to form a protein-loaded cross-linked hydrogel. The mixture can be molded or extruded to form the final delivery system or formed in situ. The cross-linking reaction proceeds via the mechanism shown in FIGS. 6A and 6B. Various other mechanisms may be used to achieve a cross-linked polymer hydrogel.

FIG. 6A illustrates an exemplary cross-linking reaction. The reactants are polyethylene oxide-amine and polyethylene oxide-succinimidyl glutarate which react to produce a product at a pH between 7.4-8. The product may be a crosslinked network with hydrolytically labile ester linkages.

FIG. 6B illustrates another exemplary cross-linking reaction. The reactants are an amine compound and NHS ester derivative to produce an amide bond compound and an NHS leaving group.

The formulation comprising the therapeutic agent may be prepared by standard techniques or methods that are well-known in the art using one or more pharmaceutically acceptable carriers or excipients. The term “pharmaceutically acceptable,” as used herein, means a substance that does not substantially interfere with the effectiveness or the biological activity of the active ingredient (or ingredients) and which is not toxic to the patient in the amounts used. Examples of pharmaceutically acceptable carriers include sterile liquids, such as water and oils, including those of petroleum, animal, vegetable or synthetic origin, such as peanut oil, soybean oil, mineral oil, and sesame oil. Aqueous carriers, including water, are typical carriers for pharmaceutical compositions prepared for intravenous administration. As further examples, saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, and ethanol. The composition, if desired, can also contain wetting or emulsifying agents, or pH buffering agents. Pharmaceutical formulation practices, carriers, and excipients are described in, e.g., Remington Essentials of Pharmaceutics (L. A. Felton ed., 2012).

The fatty component minimizes aggregation of the therapeutic agent and maintains the stability of the therapeutic complex by limiting exposure of the therapeutic agent to the aqueous media of the eye and restricting its molecular mobility within the hydrogel. The hydrogel matrix degrades over the course of weeks to months to sustain the release of the therapeutic agent.

The fatty component may comprise a variety of different characteristics to help optimize the sustained release of the therapeutic agent. For example, in some embodiments, the fatty alcohol, fatty acid, or fatty alcohol/fatty acid blend matrix may have a solubility of less than 1 μg/mL in de-ionized water at 20° C. In some embodiments, the fatty alcohol, fatty acid, or fatty alcohol/fatty acid blend matrix may comprise a melting point selected from a range between about 48° C. and about 76° C.

In some embodiments, the fatty alcohol of the therapeutic complex may be cetyl alcohol, 1-eicosanol or stearyl alcohol. In other embodiments, the fatty acid of the therapeutic complex may be palmitic acid, arachidic acid, or stearic acid.

The therapeutic complex comprising the therapeutic agent and the fatty component dispersed in the hydrogel matrix may increase the sustained release of the therapeutic agent. In the absence of the bioerodible hydrogel matrix, the therapeutic complex may release the therapeutic agent over a period that is less than 28 days.

In some embodiments, the composite implant releases the therapeutic agent over a period of one week to 12 months from implantation in an eye of a subject, while maintaining the therapeutic agent activity. In some embodiments, the composite implant releases the therapeutic agent for a period of at least six months from implantation in an eye of a subject.

In some embodiments, the composite implant may exhibit a burst release of the therapeutic agent that is less than about 10% (w/w) over an initial 24-hour period from implantation in an eye of a subject. In some embodiments, the composite implant may exhibit a burst release of the therapeutic agent that is less than about 5% (w/w) over an initial 24-hour period from implantation in an eye of a subject.

The release rate of the therapeutic agent from the composite implant may be substantially constant. For example, in some embodiments, the release rate of the therapeutic agent from the composite implant may be substantially constant over an initial three-month period starting at the end of the burst release or lag phase of the therapeutic agent, but not more than 14 days after implantation or in vitro release studies. The lag phase may be defined as the period immediately post-implantation or immediately after initiating in vitro release studies where no drug is released or the drug is released at a slower rate than the constant rate achieved after not more than 14 days.

The release rate of the therapeutic agent from the composite implant may be near zero order or pseudo-zero order. For example, in some embodiments, the release rate of the therapeutic agent from the composite implant may be near zero order or pseudo-zero order over an initial three-month period from implantation starting at the end of the burst release or lag phase of the therapeutic agent. Near zero order release and pseudo-zero order release kinetics may be defined as an essentially linear relationship between the cumulative amount of therapeutic agent released from the composite hydrogel in vivo or in in vitro release studies as a function of time.

The composite implant may be introduced, implanted, injected or otherwise delivered into an eye of a subject or a patient. The composite implant may be delivered by implanting the composite implant through a pars-plana injection into a vitreous or posterior chamber of an eye of the subject with a single use application through a needle. In some embodiments, the needle may be less than about 19 gauge, less than about 20 gauge, less than about 21 gauge, less than about 22 gauge, less than about 25 gauge, or other appropriate diameter. In particular embodiments, the needle is a 21 gauge or smaller diameter needle.

The present disclosure also provides methods related to the use of composite implants. In certain embodiments, the present disclosure provides methods of introducing a therapeutic agent into an eye of a subject. Such methods comprise delivering a composite implant to as described above into an eye of a subject. In other embodiments, the present disclosure provides methods of treating an ocular disease in a subject that comprise delivering a composite implant as described above to an eye of the subject. The ocular disease may be selected from at least one of neovascular age-related macular degeneration (AMD), diabetic macular edema, and macular edema following retinal vein occlusion.

The present disclosure also provides a therapeutic agent for use in treating an ocular disease, wherein the therapeutic agent is provided in a composite implant as described above. Furthermore, the present disclosure provides for use of a therapeutic agent in the manufacture of a composite implant as described above for treatment of a subject in need thereof. The present disclosure also provides a pre-loaded injector assembly comprising a needle and a composite implant as described above.

### EXAMPLES

To further illustrate these embodiments, the following examples are provided. These examples are not intended to limit the scope of the claimed invention, which should be determined solely on the basis of the attached claims.

### Example 1—a Bevacizumab PEG Hydrogel Composite Implant

This example describes a bevacizumab (Avastin®) PEG hydrogel composite implant without a therapeutic complex. Biodegradable hydrogels of cross-linked PEG with a lyophilized bevacizumab core were prepared according to the formulation in Table 1. Briefly, a 10 KDa 8-arm PEG-SG was added to a vial of dichloromethane. In a separate vial, a 10 KDa 8-arm PEG-NH was added to dichloromethane. A lyophilized bevacizumab core was added to the PEG-NH solution. The two vials were combined and quickly drawn up into a silicone tube to form the composite implant. The composite implant was vacuum dried to remove any residual dichloromethane. The bevacizumab core was manufactured by co-lyophilizing bevacizumab (25 mg/mL in aqueous solution) with trehalose dihydrate (60 mg/mL), monobasic sodium phosphate (5.8 mg/mL), dibasic sodium phosphate (1.2 mg/mL), and polysorbate (PS) 20 (0.4 mg/mL).

TABLE 1
Avastin PEG-SG hydrogel formulation.

| | | | % Lyophilized | % Pure |
|---|---|---|---|---|
| | PEG-Amine | PEG-NHS | Formulation | Avastin |
| Name | Reagent | Reagent | Loading | Loading |
| Sample 1 | 8-arm NH2 | 8-arm | 72.3% | 18.1% |
| | | PEG-SG | | |

Release of the bevacizumab from the implants was assessed in vitro. Implants were placed into 5 mL glass vials containing isotonic phosphate buffered saline (IPBS) at pH 7.4 as the release media. The vials were then placed on a shaker bath to agitate the medium at 37° C. At pre-determined time points, the media was sampled and the entire receiver media was replaced with fresh IPBS. The bevacizumab concentration in the sampled aliquot was quantified by HPLC using a Waters Alliance e2695 system with a C-18 BEH column. The bevacizumab concentrations were used to define the cumulative in vitro release of bevacizumab from the implant as well as the daily bevacizumab release rate.

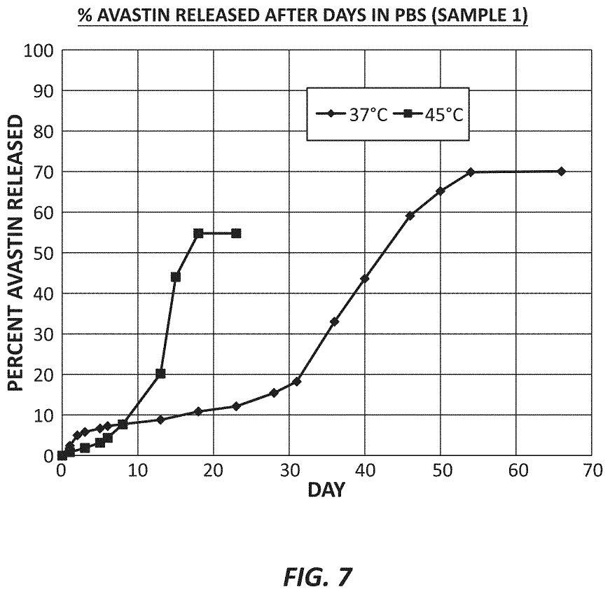

The PEG-SG was able to sustain the release of the protein for 50 days. However, beginning on day 13, aggregates began to form and protein degradation took over at day 31 as shown in Table 2. The bevacizumab release as a function of time is illustrated in FIG. 7. A first graph line occurs at 37° C. and the second graph line occurs at 45° C.

TABLE 2
Aggregates and degradants of bevacizumab in
the release media as a function of time.

| Day | % Monomer | % Aggregates | % Degradants |
|---|---|---|---|
| 0 | 100 | 0 | 0 |
| 1 | 100 | 0 | 0 |
| 2 | 100 | 0 | 0 |
| 3 | 100 | 0 | 0 |
| 5 | 100 | 0 | 0 |
| 6 | 100 | 0 | 0 |
| 8 | 100 | 0 | 0 |
| 13 | 90 | 10 | 0 |
| 18 | 90 | 10 | 0 |
| 23 | 85 | 15 | 0 |
| 28 | 85 | 15 | 0 |
| 31 | 90 | 0 | 10 |
| 36 | 75 | 0 | 25 |
| 40 | 75 | 0 | 25 |
| 46 | 50 | 0 | 50 |
| 50 | 40 | 0 | 60 |
| 54 | 40 | 0 | 60 |
| 66 | 40 | 0 | 40 |

In another experiment, bevacizumab loaded PEG hydrogels were manufactured using three different reactive PEG-NHS groups. The hydrogels were loaded with 13% bevacizumab. The PEG-NHS groups included PEG-SG, PEG-SAP, and PEG-SAZ. The erosion of the hydrogel was followed in an in vitro dissolution bath as described for the release studies above. The time for hydrogel implant erosion was noted at various temperatures: 37° C., 45° C., and 50° C. The PEG-SG 10 KDa hydrogel took 78, 29, and 21 days to erode at 37° C., 45° C., and 50° C., respectively. The PEG-SAP 10 KDa hydrogel took 156, 42, and 32 days to erode at 37° C., 45° C., and 50° C., respectively. The PEG-SAZ 10 KDa hydrogel took 613, 141, and 59 days to erode at 37° C., 45° C., and 50° C., respectively. Hence, PEG backbones have been identified to allow for protein delivery at physiologic pH over a period of two months to 1.6 years.

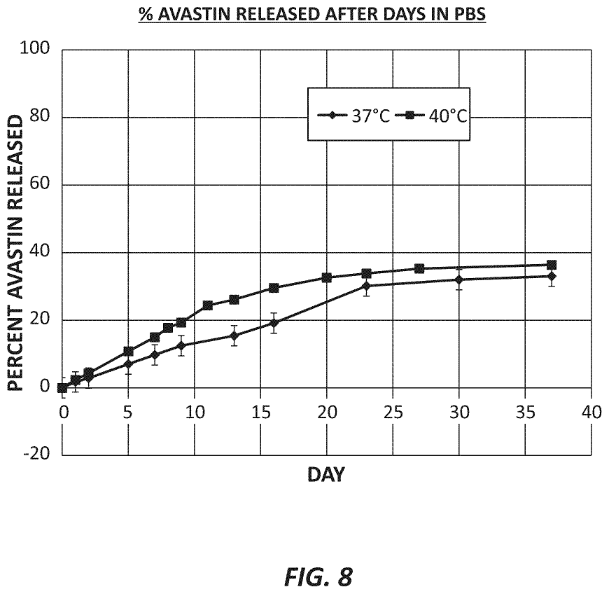

Another bevacizumab formulation was manufactured in an aqueous medium with the addition of plain PEG (1,000 D) as a method to reduce the solubility of protein during the cross-linking reaction. The formulation parameters are shown in Table 3. The bevacizumab core was manufactured by co-lyophilizing bevacizumab (25 mg/mL) in trehalose dihydrate (60 mg/mL), monobasic sodium phosphate (5.8 mg/mL), dibasic sodium phosphate (1.2 mg/mL), and PS 20 (0.4 mg/mL). The in vitro release of bevacizumab was assessed as above. Significant aggregation was noted by day 30 as shown in Table 4. The cumulative release of bevacizumab in vitro as a function of time is depicted in FIG. 8. A first graph line occurs at 37° C. and the second graph line occurs at 40° C.

TABLE 3
Bevacizumab PEG-SAP hydrogel formulation.

| | | | | % Pure |
|---|---|---|---|---|
| | PEG-Amine | PEG-NHS | % Plain PEG | Bevacizumab |
| Name | Reagent | Reagent | Loading | Loading |
| 9AVST-SAP | 8-arm NH2 | 8-arm | 29.2% | 8.9% |
| | | PEG-SAP | | |

TABLE 4
Aggregates of bevacizumab in the release
media as a function of time.

| Day | % Monomer | % Aggregates |
|---|---|---|
| 0 | 100 | 0 |
| 1 | 100 | 0 |
| 2 | 100 | 0 |
| 5 | 100 | 0 |
| 7 | 100 | 0 |
| 9 | 100 | 0 |
| 13 | 100 | 0 |
| 16 | 97 | 3 |
| 23 | 90 | 10 |
| 30 | 70 | 30 |

### Example 2—an Aflibercept PEG Hydrogel Composite Implant

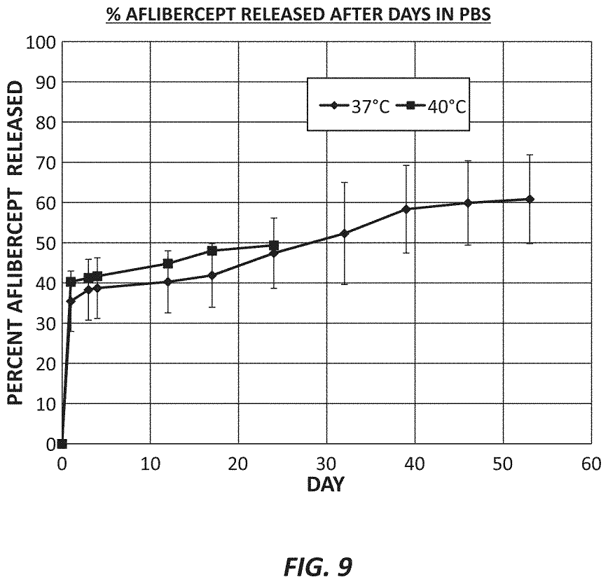

This example describes an aflibercept PEG hydrogel implant without a therapeutic complex. Biodegradable hydrogels of cross-linked PEG with a lyophilized aflibercept core were prepared by dissolving the PEG reagents in DCM according to the formulation in Table 5. The aflibercept core was manufactured by co-lyophilizing aflibercept (150 mg/mL) in a solution of sodium acetate buffer (pH 5.2, 150 mM), histidine (20 mM), arginine (150 mM), methionine (5 mg/mL), P407 (100 mg/mL), and PS 20 (0.05%). The in vitro release of aflibercept was assessed as above. The cumulative in vitro release of aflibercept as a function of time is depicted in FIG. 9. A first graph line occurs at 37° C. and the second graph line occurs at 40° C. As can be seen in Table 6, significant aggregation of aflibercept begins on day 39.

TABLE 5
Aflibercept hydrogel formulation

| | | | % Aflibercept | % Pure |
|---|---|---|---|---|
| | PEG-Amine | PEG-NHS | Formulation | Aflibercept |
| Name | Reagent | Reagent | Loading | Loading |
| 5AFSP | 4-arm | 8-arm | 30.2% | 15% |
| | PEG-NH2 | PEG-SAP | | |
| | 10 KDa | 5 KDa | | |

TABLE 6
Aggregates and degradants of aflibercept in
the release media as a function of time.

| Day | % Dimer | % Aggregates |
|---|---|---|
| 0 | 100 | 0 |
| 1 | 100 | 0 |
| 3 | 100 | 0 |
| 4 | 100 | 0 |
| 12 | 100 | 0 |
| 17 | 100 | 0 |
| 24 | 100 | 0 |
| 32 | 97 | 3 |
| 39 | 88 | 12 |
| 46 | 35 | 65 |

Manufacture without loss of protein activity and release over 30 to 60 days was achieved. However, both bevacizumab and aflibercept began aggregating and degrading after 30 days in the cross-linked non-composite PEG hydrogels.

### Example 3—Fatty Alcohol Microparticulates

This example describes fatty alcohols without the PEG hydrogel. Associating proteins with a fatty component alone failed to provide sustained delivery. All protein was released within the first day when stearyl alcohol was used. For example, aflibercept-loaded complexes comprising stearic acid or stearyl alcohol were manufactured co-lyophilzation. The in vitro release of aflibercept from these complexes was assessed as per above. All protein was released within the first day. The fatty alcohols and fatty acids on their own are insufficient to achieve sustained release of proteins to the eye.

### Example 4—Composite Systems

This example describes composite systems that include therapeutic complexes of aflibercept associated with fatty alcohols.

Biodegradable hydrogels of cross-linked PEG with a lyophilized aflibercept complex prepared according to the formulation in Table 7. The complex was prepared using (i) a protein and (ii) cetyl alcohol or 1-eicosanol. Incorporating the protein into the fatty alcohol involved dispersing the fatty alcohol in water, adding an aqueous solution of the protein, and lyophilizing the mixture. FIG. 10 depicts exemplary composite hydrogel implants.

TABLE 7
Aflibercept composite hydrogel formulation.

| | PEG-Amine | PEG-NHS | | % Solids | Aflibercept |
|---|---|---|---|---|---|
| Name | Reagent | Reagent | Core FA | Loading | Loading |
| Sample 2 | 8-arm | 8-arm | Cetyl | 32.7% | 15.0% |
| | PEG-NH2 | PEG-SG | alcohol | | |
| | 10 KDa | 10 KDa | | | |
| Sample 3 | 8-arm | 8-arm | 1-eicosanol | 32.7% | 15.0% |
| | PEG-NH2 | PEG-SG | | | |
| | 10 KDa | 10 KDa | | | |
| Sample 4 | 8-arm | 8-arm | 1-eicosanol | 37.8% | 15.0% |
| | PEG-NH2 | PEG-SAP | | | |
| | 10 KDa | 10 KDa | | | |

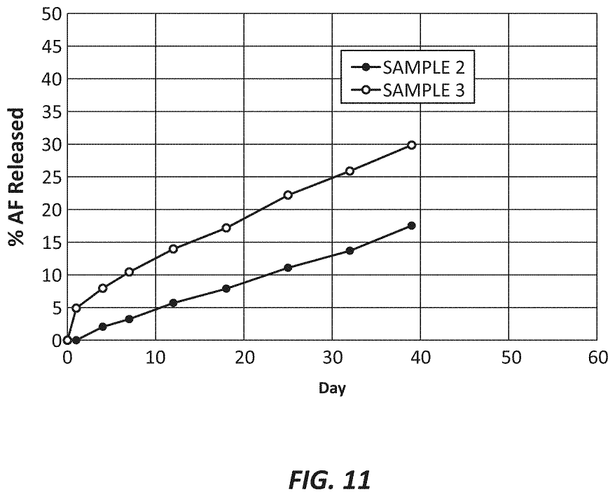

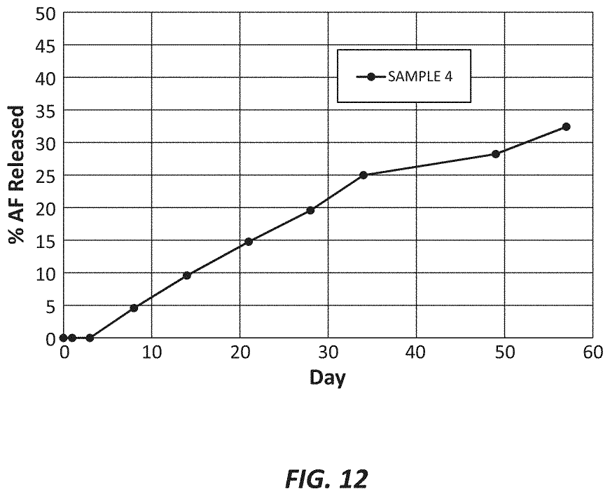

The release of aflibercept from the composite hydrogels was assessed as above. The release profiles are depicted in FIGS. 11 and 12. FIG. 11 illustrates the release profile of sample 2 and sample 3 whereas FIG. 12 illustrates the release profile of sample 4.

The PEG/fatty alcohols (PEG/FA) reduced aflibercept burst significantly and demonstrated controlled release. The PEG-SG/FA gels erode in 30-40 days. PEG-SAZ/FA gels last six months. Further advantages of using a therapeutic complex of a therapeutic agent and a fatty component is the ability to use aqueous manufacturing to prepare said complexes and/or composite systems, thereby avoiding harsh and potentially unsafe organic solvents. Unique attributes to this composite system include: stabilization of protein during in vitro and in vivo release; a pseudo-zero order release rate of protein from the composite hydrogel; a protein burst release less than 10% over the first day; and the ability to release stabile protein while maintaining stability and activity over a period of six months.

Entrapping the fatty alcohol complex in a cross-linked hydrogel matrix extends the duration of release from weeks to months. With no hydrogel to sequester the complexes, the fatty components disassociate from the therapeutic agents in a matter of hours to days. Thereby, while fatty components maintain stability of the associated therapeutic agent, they do not sustain its release. Rather, the combination of the hydrogel with the fatty alcohol particles determines the primary rate of release. Forming the hydrogel involves reacting PEG-amine with PEG-SG, PEG-SAP, and/or PEG-SAZ monomers to form cross-links. Each chemistry has its own cross-link density and subsequent rate of degradation, and so selecting the composition of the reactive PEGs allows for tuning of the biodegradation of the hydrogel, and release of its contents.

Also affecting the primary release rate are the molecular weights of the PEG monomers and the cross-linking density. 4-arm and 8-arm PEG monomers with molecular weights of 5 kDa or 10 kDa are selected here.

Somewhat surprisingly, the fatty alcohol particles appear to secondarily affect the release by minimizing burst, wherein a substantial portion of protein is released from the hydrogel in the first hours to days following aqueous exposure.

More recent methods use organic solvents when forming protein-entrapping hydrogels in order to prevent exposure of the protein to water. In essence, the protein remains out of solution and in a more stable solid state. Direct exposure to these organic solvents may cause harm to the protein, though, and cross-linking reactions occur much more quickly in preferred organic solvents, limiting the working time and presenting an obstacle to scale-up of the method when such delivery systems reach market. Hence, forming protein-entrapping hydrogels in an aqueous solution may be advantageous. In the absence of fatty alcohols or fatty acids, we observe disruption to the cross-linking reaction in aqueous media as protein enters the solution prior to gel formation, resulting in a poorly formed hydrogel. FA particles allow the cross-linking reaction to proceed by protecting the protein from direct exposure to the aqueous solution, thereby achieving a similar effect as the organic solvents method with a working time more feasible to scale.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

We claim:

1. A composite implant comprising:
a bioerodible cross-linked polyethylene glycol hydrogel; and
a therapeutic complex comprising:
a therapeutic agent; and
a fatty component;
wherein the therapeutic complex is dispersed in the bioerodible cross-linked polyethylene glycol hydrogel.

2. The composite implant of claim 1, wherein the composite implant is configured to be delivered to or implanted in an eye of a subject.

3. The composite implant of claim 1, wherein the bioerodible cross-linked polyethylene glycol hydrogel comprises a network of polyethylene glycol formed by a reaction between a polyethylene glycol with an electrophilic end group and polyethylene glycol with a nucleophilic end group.

4. The composite implant of claim 3, wherein the electrophilic end group comprises a hydroxysuccinimidyl glutarate (SG), an N-hydroxysuccinimidyl adipate (SAP), or an N-hydroxysuccinimidyl azelate (SAZ).

5. The composite implant of claim 1, wherein a burst release of the therapeutic agent from the composite implant is less than about 10 percent (w/w) over an initial 24-hour period from implantation in an eye of a subject.

6. The composite implant of claim 1, wherein a burst release of the therapeutic agent from the composite implant ranges from between about 0 and about 5 percent (w/w) over an initial 24-hour period from implantation in an eye of a subject.

7. The composite implant of claim 1, wherein the release rate of the therapeutic agent from the composite implant is substantially constant over an initial three-month period beginning with the end of the burst release or lag phase, but not more than 14 days post-implantation.

8. The composite implant of claim 7, wherein the release rate of the therapeutic agent from the composite implant is near zero order or pseudo-zero order over an initial three-month period from implantation beginning with the end of the burst release or lag phase, but not more than 14 days post-implantation.

9. The composite implant of claim 1, wherein the composite implant releases the therapeutic agent for a period of at least six months from implantation in an eye of a subject.

10. The composite implant of claim 1, wherein the fatty component comprises a fatty alcohol.

11. The composite implant of claim 10, wherein the fatty alcohol is cetyl alcohol, 1-eicosanol or stearyl alcohol.

12. The composite implant of claim 1, wherein the fatty component comprises a fatty acid.

13. The composite implant of claim 12, wherein the fatty acid is palmitic acid, arachidic acid, or stearic acid.

14. The composite implant of claim 1, wherein the therapeutic agent is selected from at least one of a protein, a peptide, a nucleic acid, an RNA, an siRNA, an apatamer, or a small molecule.

15. The composite implant of claim 1, wherein the therapeutic agent is selected from at least one of a prostaglandin, a neuroprotectant, a retinoid, squalamine, a steroid, an alpha adrenergic agent, a gene, an antibiotic, a non-steroidal anti-inflammatory agent, a calcineurin inhibitor, an adeno-associated virus vector, a tyrosine kinase inhibitor, or a rho kinase inhibitor.

16. The composite implant of claim 1, wherein the therapeutic agent is selected from at least one of bevacizumab, ranibizumab, aflibercept, brolucizumab, faricimab, conbercept, ankyrin repeat proteins, adalimumab, anti-TNF-alpha agents, biosimilars, or salts, esters, solvates, isomers, complexes, or conjugates thereof.

17. The composite implant of claim 1, wherein the therapeutic agent is associated with the fatty component by at least one of being dispersed within the fatty component, coated by the fatty component, adsorbed by the fatty component, or a combination thereof.

18. A method of introducing a therapeutic agent into an eye of a subject, the method comprising:
delivering a composite implant to an eye of a subject, the composite implant comprising:
a bioerodible cross-linked polyethylene glycol hydrogel; and
a therapeutic complex comprising:
a therapeutic agent; and
a fatty component;
wherein the therapeutic complex is dispersed in the bioerodible cross-linked polyethylene glycol hydrogel.

19. The method of claim 18, wherein the delivering a composite implant to an eye of a subject comprises injecting the composite implant through a pars-plana injection into the vitreous or posterior chamber of an eye of the subject.

20. A pre-loaded injector assembly comprising:
a needle; and
a composite implant comprising:
a bioerodible cross-linked polyethylene glycol hydrogel; and
a therapeutic complex comprising:
a therapeutic agent; and
a fatty component,
wherein the complex is dispersed in the bioerodible cross-linked polyethylene glycol hydrogel, and
wherein the composite implant is loaded in the needle.
